(19) United States
(12) Patent Application Publication
(10) Pub. No.: US 2002/0054917 A1
(43) Pub. Date: May 9, 2002

(54) Compositions comprising beta glucan and lactoferrin, and methods for their use

(76) Inventor: Marcus B. Gohlke, Houston, TX (US)

Correspondence Address: R. William Beard, Jr., Baker Botts L.L.P., 910 Louisiana Street, Houston, TX, 77002-4995, US

(21) Appl. No.: 10/021,970

(22) Filed: Dec. 13, 2001

Related U.S. Application Data
(63) Continuation-in-part of application No. 09/778,294, filed on Feb. 6, 2001.
Application No. 09/778,294 is a division of application No. 09/370,654, filed on Aug. 6, 1999.
(60) Provisional application No. 60/096,697, filed on Aug. 14, 1998.
(60) Provisional application No. 60/257,013, filed on Dec. 20, 2000.

(51) Int. Cl. A61K 35/20; A61K 31/715; A01N 43/04

(52) U.S. Cl. 424/535; 514/54; 514/59

(57) ABSTRACT

Dietary supplement compositions containing beta glucan and lactoferrin are disclosed. The compositions can be formulated as a liquid, tablet, lozenge, gum, or other acceptable forms. The compositions can further contain flavorings, carriers, or diluents. The compositions can be used in a nutritional program to enhance the activity of macrophages and neutrophils, leading to an improved ability to fight infection from bacterial, fungal, and viral challenges.

### CROSS REFERENCE TO RELATED APPLICATIONS

The present application is a continuation-in-part (CIP) of co-pending U.S. patent application Ser. No. 09/778,294, filed Feb. 6, 2001, which is a divisional of U.S. patent application Ser. No. 09/370,654), filed Aug. 6, 1999 (issued as U.S. Pat. No. 6,258,383), which claims the benefit of U.S. Provisional Patent Application Serial No. 60/096,697, filed Aug. 14, 1998. The present application also claims the benefit of U.S. Provisional Patent Application Serial No. 60/257,013, filed Dec. 20, 2000.

### FIELD OF THE INVENTION

The invention relates to natural dietary supplements and their use. More specifically, it relates particularly to dietary supplements that enhance the activity of the two major classes of white blood cells, namely macrophages and neutrophils. This accentuates the host's natural ability to overcome metabolic insults. In particular, compositions comprising beta glucan and lactoferrin are disclosed.

### BACKGROUND OF THE INVENTION

Nutrition is a critical determinant of immunological competence and of the individual's ability to fight infection from bacterial, viral, or fungal sources, cancer development and to slow the lethal effects of septicemia (sepsis). The health of individuals affected by viral infections, general bacterial infections, antibiotic resistant bacterial infections, fungal infections, cancer, and the proliferation of any bacteria in the blood (septicemia) cannot be overlooked. In particular, pollution, food additives, toxins, the routine use of antibiotics, and bioterrorism have resulted in multiple adverse influences upon health. Among these may be included the proliferation of new, more deadly strains of bacteria and viruses that are resistant to existing treatment, compromised immune systems resulting from chemical pollutants in food, water and air. The continuing widespread use of man-made chemicals in medicine and agriculture reinforces the selective pressures that increase the types and extent of antibiotic-resistant microbes in the environment, which then substantially increases the cost of treating infection.

Additionally, emotional and physical stresses from the natural effects of aging reduce the effectiveness of the immune system and its role in overcoming these adverse agents. The physiological rigors to which an individual's body is exposed in the modem environment, which include the chemical pollutants and antibiotic-resistant microbes discussed above, indicate the advisability of boosting the immune system's white blood cell interaction to facilitate the body's abilities to resist and cope with infection, and to assist the natural, self-healing processes. Two groups of individuals are particularly susceptible to infection and the side effects of treatment: young children and the aged. These individuals may respond poorly to physiological or environmental challenges because they typically possess immune systems that are, in young children and in the aged respectively, immature or damaged. Consequently, natural fortification of these individuals' white blood cells is particularly desirable.

The importance of macrophages and neutrophils, the two major classes of white blood cells, is well documented. The basis for elimination of cancer cells, fighting infection, and the prevention and treatment of septicemia is contingent upon the interaction of macrophages and neutrophils. The neutrophil immediately goes to the site of an insult; the outer membrane will burst and then release lactoferrin. The lactoferrin sequesters any free iron available for the pathogens growth as well as starts to degrade the pathogen's outer membrane. In doing so, it marks the intruder for the macrophage to destroy. Without the assistance from the neutrophil the macrophage is severely limited in its efficacy, therefore an abundance of neutrophils along with enhanced macrophages results in much faster elimination of cancer cells, infections, and slowing the progression of any bacterial infection into a lethal toxin cascade that ultimately results in septicemia

Stimulation of the immune system may occur if the appropriate glycoproteins and proteins are absorbed into an individual's bloodstream. These biomolecules are expensive to obtain, even in the quantities and formats used for experimental demonstrations (Lonnerdal & Iyer, Annu. Rev. Nutr., 15: 93-110, 1995). Attempts to formulate an effective dietary supplement able to generate and maintain a state of white cell equilibrium in an individual have been unsuccessful, leading to the need for the wide variety and potency of antibiotics.

If the components of a dietary supplement were to possess, in addition to nutritional characteristics, abilities that aid the body's ability to eliminate cancer cells, fight infection, and prevent and treat septicemia, such abilities would naturally prove advantageous for achieving the health and well being enhancing the purposes outlined above.

As indicated below, those skilled in the art of the respective fields recognize that beta glucan and lactoferrin, are individually able to perform limited beneficial activities of this type (Wang, et al., J. Leuk. Biol., 75: 865-874, 1995; and Burrin et al., Pediatr. Res., 37: 593-599, 1995).

Beta glucan is a complex sugar (polysaccharide). Beta glucan can be obtained from a wide array of sources such as baker's yeast, fungal cells, mushrooms, barley, and oats. Beta glucans can be characterized by their main chain linkages and by their branching at various positions in the saccharose rings. Beta 1,3 D-glucan, Beta 1-3, 1-4 D-glucan, and Beta 1-3, 1-6 D-glucan are some of the more common types found. Naturally obtained beta glucan contains a mixture of all of the chains in different proportions. For instance, a mixture may have more Beta 1,3 linkages than 1-4 and 1-6 linkages. Different organisms produce beta glucans with different types and distributions of linkages. Most of the scientific literature focuses on the most common form, Beta 1,3 D-glucan. As used herein, “beta glucan” refers to mixtures that can contain one, two, or all three variations. Beta glucans can further be chemically modified, e.g. by sulfation, amination, and cross-linking.

Research has been reported on some of the beneficial properties of beta glucan. Macrophage activation was reported in vitro using a cross-linked 1,3 beta glucan (Adachi, Y., et al. Chem. Pharm. Bull. (Tokyo) 38: 988-992, 1990). Glucan activated macrophages were reported to enhance host resistance to malignancies (DiLuzio, N. R. et al., The Macrophage in Neoplasia, Academic Press, Inc., 181-198, 1976; Artursson, P. et al., Scand. J. Immunol., 25(3): 245-254, 1987; Reynold, J. A. et al., Infection and Immunity, 30: 51, 1980). Anti-tumor and anti-cancer effects of glucans have been reported (Bomford, R. and Moreno, C., Br. J. Cancer, 36: 41-48, 1977; Bomford, R. and Moreno, C. Dev. Biol. Stand., 38: 291-295, 1977; Morikawa, K., et al., Cancer Res. 45: 1496-1501, 1985; Proctor, J. W. et al., Cancer Treat. Rep. A2(11): 1873-1880, 1978; Proctor, J. W. and Yamamura, Y., J. Natl. Cancer Inst. 61: 1179-1180, 1978). The anti-sepsis properties of glucans have also been reported (Tsujinaka, T. and Yokota, M. K., Euro. Surg. Res., 22: 540-546, 1990; Rasmussen, L. T. and Seljelid, R., Scand. J. Immunol. 32(4): 333-340, 1990; Lahnborg, G. et al., J. Reticuloendomenal Soc., 32: 347-355, 1982; Lahnborg, G. et al., Eur. Surg. Res., 401-408, 1982)

Lactoferrin is a protein that is secreted in milk, tears, mucus and saliva, and is expressed by white cells at the site of attack by numerous pathogens. A primary function of lactoferrin is to bind iron at the molecular level and thereby act as a highly effective antimicrobial agent. Iron is an essential growth factor for virtually every cell and microorganism, and free iron promotes the growth of pathogens in the intestines (bacteria, fungi, and viruses), permitting invasion of the rest of the body through the intestinal walls (Gillon Ward et al., J. Trauma, Inj. Inf. Critical Care, 41: 356-364, 1996). Lactoferrin is released by neutrophils to absorb free iron that would otherwise be available to bacteria, viruses and fungi for growth. Unlike synthetic antibiotics, to which bacteria may develop resistance through mutation, lactoferrin exerts its bacteriostatic effect as long as the bacteria require iron for growth.

Additionally, lactoferrin is recognized by specific receptors in mammalian tissues, and upon binding, releases iron to the body for normal, healthy cell growth. Unlike synthetic antibiotics, lactoferrin has the ability to bind iron, transport it and then release the iron specifically to the body's own cells through cell surface lactoferrin receptors. Binding of bovine lactoferrin to B. bifidum and B. breve was about 40-fold higher than binding to Escherichia coli, regardless of the iron saturation level of the lactoferrin (Petschow B W, et al., J. Med. Microbiol., 48(6):541-549, 1999).

Lactoferrin is a multifunctional protein that is produced in a variety of cell types under different mechanisms of control. It has been demonstrated that lactoferrin plays a central role in the inflammatory defense processes. Released in abundant quantities by neutrophils attracted to the site of an invasion, lactoferrin binds the iron made available by serum and damaged erythrocytes. Monocytes and macrophages ingest the iron-saturated lactoferrin, which has also been implicated in the production of metastable oxygen metabolites associated with bacterial destruction within these blood cells (Wang, et al., J. Leuk. Biol., 75: 865-874, 1995). Lactoferrin also regulates the release of tumor necrosis factor alpha (TNF-alpha) and interleukin 6 (IL-6) in vivo (Machniki et al, Int. J. Exp. Path., 74: 433-439, 1993). Lactoferrin increases the number of fresh neutrophils in circulation by up to 116% (Zimecki M, Arch. Immunol. Ther. Exp. (Warsz), 47(2):113-118, 1999).

Due to the iron absorption and release functions of this protein, lactoferrin is the body's primary regulator of iron, a major bio-regulator of the digestive tract and a natural bacteriostatic agent having indirect but broad antibiotic effects. Yet the cost and availability of human lactoferrin, purified from human breast milk, restricts its use to research.

Lactoferrin's iron-binding bacteriostatic effect, coupled with its general abundance in breast milk, has led to numerous studies in new-born mammalian offspring, prompting its incorporation into Japanese baby formula since approximately 1993. Lactoferrin B is an amino terminal peptide of bovine lactoferrin generated by pepsin digestion and has been shown to have a potent bactericidal activity against a diverse range of potentially pathogenic bacteria (Bellamy et al, J. Applied Bacteriol., 73: 472-479, 1992). The importance of lactoferrin in newborn humans for ensuring the appropriate formation and development of the gastrointestinal tract, its bacterial colonization and to enable nutrients to be absorbed effectively has also been demonstrated.

Many of these functions of lactoferrin are reviewed by Lonnerdal & Iyer (Annu. Rev. Nutr., 15: 93-110, 1995). Yet these authors note that the relative efficacy of using either lactoferrin from other species, or recombinant human lactoferrin for treatment of humans is unproven. This is because adequate quantities of human lactoferrin have not been isolated to supply clinical studies, and recombinant human lactoferrin will not accurately reproduce the protein's glycan composition.

Tanaka et al., U.S. Pat. Nos. 5,098,722 and 5,008,120 suggest methods of preparing iron-fortified beverages that contain a solution of purified bovine lactoferrin and provide high bio-availability of iron.

Tomita et al., U.S. Pat. No. 5,304,633 presents fragments of milk lactoferrin having potent antimicrobial activity. Kunio et al., U.S. Pat. No. 5,576,299 suggests the use of lactoferrin for preventing and treating the opportunistic infections that arise in immuno-compromised individuals. Yamamoto et al., U.S. Pat. No. 5,725,864 offers the use of an iron-binding protein, of which lactoferrin is one of several examples, for inhibiting infection or suppressing growth of human immunodeficiency virus. The protein is administered by diffusion through any of several epithelial membranes, or by injection. Valenti & Antonini, U.S. Pat. No. 5,834,424 proposes the use of compositions containing lactoferrin or other iron-binding proteins for treating Gram-positive bacterial infections.

Nichols & McKee, U.S. Pat. No. 4,977,137 suggests the use of milk lactoferrin from human and other mammalian sources as a dietary ingredient or supplement. The lactoferrin promoted growth of the gastrointestinal tract of human infants or non-human animals immediately on birth. Konig et al., U.S. Pat. No. 5,466,669 offers an immunostimulatory agent comprising a peptide derived from lactoferrin.

Headon et al., PCT/US90/02356 and European Patent No. 0 471 011 B1 presents the verified cDNA sequence of human lactoferrin. Kruzel, PCT/US91/01335 offers human lactoferrin expressed from recombinant DNA, its method of production and purification and its use for supplementing the diet with trace elements or as a topical antiseptic. Kruzel et al., PCT/US95/05653 discusses the cloning, expression and uses of recombinant human lactoferrin for retarding food spoilage, as a topical antiseptic, for inhibiting microbial growth in or on a mammal, for regulating iron levels within a mammal or for a nutritional supplement.

The strongly acidic conditions of the stomach, and the function of the proteolytic enzymes and zymogens produced in the pancreas and acting in the intestines, are well known to inactivate and degrade the delicate structures of proteins, such as the components of the dietary supplements described here. The species-specific glycosylation of lactoferrins from different mammalian sources may provide protection from proteolysis for lactoferrin ingested naturally from maternal milk, and cross-species administration of lactoferrin would be expected to be far less effective (Lonnerdal & Iyer, Annu. Rev. Nutr., 15: 93-110, 1995). Even if the lactoferrin succeeds in reaching the small intestine intact, specific lactoferrin receptors enable human lactoferrin to deliver iron to the mucosal cells of human small intestine, whereas bovine lactoferrin is incapable of doing so (Cox et al., Biochim. Biophys. Acta, 558: 129-141, 1979).

The gastric survival of bovine lactoferrin has been studied (Troost F J, et al., J. of Nutrition, Aug;131(8):2101-2104, 2001). Gastric survival of bovine lactoferrin, analyzed by gel permeation chromatography under denaturing conditions, was only 62%. Buffering with a gastric pH buffer only increased gastric survival to 64%. Surface plasmon resonance analysis indicated that bovine lactoferrin binds more strongly to salivary agglutinin, especially to high molecular mass glycoprotein, which is a component of the agglutinin (Mitoma M, et al., J. Biol. Chem., 276(21):18060-18065, 2001). Mitoma demonstrated that the binding of bovine lactoferrin to salivary agglutinin was thermostable, and the optimal pH for binding was 4.0. Bovine lactoferrin binding with salivary agglutinin in the mouth results in a more stable lactoferrin component. Mucosal delivery in the mouth is preferred over the gastric delivery of lactoferrin. Accordingly, gastric delivery in the form of an enteral feed preparation or the swallowing of a capsule requires approximately 150% of the amount of lactoferrin that could be delivered directly into the mouth.

In spite of the knowledge of the beneficial properties of either beta glucan or lactoferrin when used individually, there remains a continuing need for an economical dietary supplement to balance the body's own defense and to provide for white blood cell homeostasis. The components of the supplement must be obtainable from economic and abundant sources, yet remain effective for administration to humans, and preferably to a broad range of recipient mammals. Moreover, such a dietary supplement must be absorbed effectively, without the degradation of protein constituents that is associated with regular digestive processes such as the destruction of delicate immunoglobulins by acids in the stomach.

The oral cavity contains a plethora of mechanisms to counter the survival of infectious agents that enter through the mouth and nose: secreted with the saliva are broad-spectrum IgA antibodies, lysozyme, and small quantities of lactoferrin, and lymphoid cells entering the oral cavity through the gingiva. In addition, it has recently been recognized that external factors may also deliver signals that modulate immune responses: these factors include cytokines such as the interferons, as well as hormones, growth factors and cellular antigens.

Studies aimed at preventing allergic inflammation in rodents have indicated that administration of interferon to mice by oral feeding could be as effective as intraperitoneal injection. Thus oral administration of either antigens or cytokines may be capable of modulating a variety of physiological reactions, including immune responses. Possible routes of mediation are: (a) taste buds of the tongue, connected by nerves to hypothalamus collateral centers, control appetite and energy utilization; (b) a spectrum of mucosal and secretory cell types present in the oral cavity that are capable of responding to cytokine or antigen signals and releasing further cytokine messages; (c) epithelial cells of the oral cavity, which are likely to be the natural recipients of signals entering the mammalian mouth: in the adult these would be primarily signals from antigens, whereas for the neonatal mammal important signals would also be received from ingested maternal cytokines and maternal hormones; (d) the submucosal tissue of the oral cavity, which secretes immunoglobulin IgA. Small amounts of either cytokine or antigen may be recognized as antigen by a responsive cell, resulting in immune activation via initiation of the cytokine cascade, whereas large doses or extended administration may induce tolerance: studies have shown that interferon administered in large doses to humans may be less effective than minimal quantities. Thus the response will frequently be individual or case dependent and may be strongly influenced by additional physiological or environmental factors.

The implications of these immunological studies have been reviewed recently (Georgiades, Biotherapy, 11: 39-51, 1998) with the conclusion that the tolerance phenomenon is not only limited to the oral administration of antigen but may occur when immunization is attempted via any mucosal membrane, such as the nasal tract.

Thus, despite the research reported to date, there still exists a need for novel nutritional supplement compositions which are effective at stimulating macrophages and neutrophils to achieve a balanced attack on pathogens.

### SUMMARY OF THE INVENTION

Nutritional supplement compositions comprising beta glucan and lactoferrin are disclosed. The compositions can further comprise nutritionally acceptable carriers, diluents, or flavorings. The compositions can be used in a nutritional program to enhance the activity of macrophages and neutrophils, improving the body's ability to fight infection from bacterial, viral, or fungal challenges. The administration of a combination of beta glucan and lactoferrin affords a balanced effect of activated macrophages and an increased number of neutrophils that would not be achieved by the administration of either material alone.

### DETAILED DESCRIPTION OF THE INVENTION

In the light of the conflicting results and controversial hypotheses discussed above, it could be considered counter-intuitive, and certainly unpredictable, to attempt to stimulate or potentiate an immune response by administering cytokines and immunoglobulins orally by means of a combination of beta glucan and lactoferrin.

The present invention is generally directed towards dietary supplements containing beta glucan and lactoferrin, thus providing an improvement over previous supplements which lacked one or other or both of these components. When absorbed in combination, the effects of beta glucan and lactoferrin on the health and well-being of the recipient are surprisingly beneficial, being greater than would be anticipated from the known properties of each component taken in isolation and including the elimination of cancer cells and tumors, enhancing the body's attack against viral infections, general bacterial infections, antibiotic resistant bacterial infections, and arresting the proliferation of any bacteria in the blood thereby reducing the occurrence of septicemia. Either delivery of the components in the gastro-intestinal tract via a dose specific capsule or absorption of the components in the oral cavity is particularly efficacious: hence the invention includes capsules or the provision of the components in a mucosal delivery format (“MDF”) such as a chewable lozenge.

Numerous components can solicit responses by the white blood cells; however, only a combination of beta glucan and lactoferrin can enhance both groups of cells and achieve a desired “balanced” effect. The inventive compositions enhance the potency of macrophages along with the number of fresh neutrophils in circulation. This enhancement and balancing of the white blood cell group results in optimum infection fighting, cancer cell reduction, and the elimination of septicemia. The enhancement and balanced effects would not be achived by administration of either component by itself.

The present invention provides a dietary supplement comprising beta glucan and lactoferrin. For mucosal delivery format, it also provides a composition containing these ingredients, which may also include nutritionally acceptable carriers, diluents and flavorings, a method of administering such a composition in a form appropriate for absorption through the lining of the oral cavity, and a method of eliminating cancer cells and tumors, enhancing the body's attack against viral infections, general bacterial infections, antibiotic resistant bacterial infections, and arresting the proliferation of any bacteria in the blood as an effective preventative and treatment against septicemia.

Providing the dietary supplement in the form of lozenges which may be dissolved slowly in the mouth may lead to more rapid effects of energizing the white blood cells naturally to keep pathogens in check. Taking a dose of the inventive composition preferably 1-3 times per day, as needed, provides the suggested dietary supplement.

The invention addresses the requirement for an effective and economical dietary supplement comprising one or more natural stimulators of immune function, elimination of cancer cells, fighting infection, and the prevention and treatment of septicemia. Furthermore, this supplement can be provided in a convenient format that permits absorption of the active components into an individual's bloodstream in a manner that avoids the body's normal digestive mechanisms. Additionally, when mucosal delivery is not available do to the age, condition, or allergy tolerances of the patient, a dose specific capsule can be used to still achieve a beneficial response. The present invention uses both gastrointestinal delivery and emphasizes the efficacy of oral administration of the dietary supplement and promotion of the supplement's efficient absorption through the oral cavity's epithelial lining by presenting the supplement in a mucosal delivery format (“MDF”). Those MDFs of the invention that are presently preferred, e.g. chewable lozenges, also render the dietary supplement of the invention particularly adaptable to self-monitored dosages, and are especially appropriate for regimes of self administration.

The invention has been found effective for numerous physiological disorders caused by and resulting in a variety of metabolic insults, including routine antibiotic use, toxic pollutants, food additives, stress, and aging.

Beta glucan from yeast has the highest concentrations of complex polysaccharides or polyglucose. Common baker's yeast (Saccharomyces cerevisiae) presently appears to have the greatest benefit, however other beta glucans are found in a variety of fungal cells, including such sources as Maitake mushroom, reishi mushroom, sacred mushroom tea, barley, and oats. Beta glucan attaches to the receptor site on the macrophage, and in doing so activates the macrophage. This provides the host with enhanced protection against viruses and bacteria and other health threats. As the body ages, macrophages become weakened and are much more difficult to stimulate. Supplemental use of this polysaccharide activates the macrophage population and enables the host to overcome metabolic insults.

Lactoferrin is an iron binding protein that occurs naturally in the body. It is secreted in milk, tears and saliva, and is expressed by white blood cells. Lactoferrin is a biological regulator that performs many important functions in the body. These functions include maintaining a healthy balance in the digestive tract, helping the immune system and promoting healthy cell growth. Dairy cattle currently provide the only cost-effective source of lactoferrin for inclusion into a dietary supplement, even though cows' milk contains a relatively low concentration of lactoferrin. Lactoferrin from cows' milk can be prepared free of lactose; it bioregulates iron, boosts the immune system, balances the digestive tract, increases energy and stamina and promotes cell growth and healing. These broad, beneficial properties are surprising in view of the inability of bovine lactoferrin to bind to the lactoferrin receptors at the surface of the mucosal cells of human small intestine.

Natural lemon flavor is a further optional component that may be incorporated to promote salivation and to adjust the acidity of the composition in order that solubility, activity and absorption of the components within the oral cavity is enhanced.

Iron is a key mineral required by all microorganisms for maintenance and growth. Excess iron in the intestines promotes pathogen growth and proliferation. Lactoferrin from cows' milk is partially saturated with iron (approximately 25% of total saturation) providing a dietary source of iron as well as a means of scavenging free iron from the oral cavity and digestive tract. Lactoferrin works on contact to starve pathogens of iron so that the correct balance of beneficial bacteria develops and is maintained in the digestive tract; the growth of harmful bacteria that are poorly adapted to these conditions being inhibited. By sequestering iron and delivering it for use by the cells of the body's internal tissues lactoferrin improves digestion and boosts the body's natural defense mechanisms. This generates more energy and increased stamina for physical activities and optimum health.

Beta glucan and lactoferrin are presently believed to achieve their optimal effects when dissolved slowly in the mouth, rather than being swallowed directly in the form of a pill or capsule. Slowly dissolving the beta glucan and lactoferrin in the mouth permits their absorption into the capillaries at the surface of the oral cavity's lining, and this is able to occur before the beta glucan and lactoferrin are exposed to the harsh degradatory conditions of the stomach and intestines. For example, bovine lactoferrin is less resistant to degradation in the human digestive tract than is human lactoferrin, and the lactoferrin receptors in the small intestine of humans will not bind bovine lactoferrin. Thus, administration of bovine lactoferrin to humans in a mucosal delivery format, such as a format that enables its absorption through the lining of the mouth, is particularly efficacious. As much as 10% of beta glucan is micronized, thereby allowing more rapid absorption where it can pass directly into the blood through the inner mucosal layer of the mouth.

Lozenges, in contrast to pills, provide a mucosal delivery format (“MDF”) for constituents (such as beta glucan and lactoferrin) which can be absorbed through the oral mucosal surface. In particular, the lozenges of the instant invention are able to enhance the benefits associated with absorption of appropriate constituents through the oral epithelial mucosa and into the underlying lymphatic system, for they are designed to be dissolved slowly in the mouth and they may also be chewable: such lozenges are therefore a presently preferred MDF. By using a cold-pressing technique to manufacture the lozenges heat degradation of sensitive biological components is minimized. Lozenges are also presently preferable to hard-pressed tablets, for the latter do not dissolve until exposed to the gastric juices of the stomach. Oral administration using lozenges as the mucosal delivery format, but not capsules or hard tablets, allows the lactoferrin to sequester iron in the upper digestive tract and thereby broaden the effect of its bacteriostatic actions.

The present invention incorporates ingredients derived from yeast and dairy sources. Given the species-specificity of human intestinal lactoferrin receptors and the apparent ease with which antigenic tolerance can be induced in a variety of mammals from rodents to primates, the efficacy of the instant invention in achieving its stated aims is remarkable and unexpected. By administering a combination of yeast beta glucan and bovine lactoferrin, not only are beneficial effects of each component observed, but a synergistic effect is apparent: the results of combined administration are greater than may be accounted for by an additive effect of the individual components. The results observed, and described below in the Examples, may stem not only from the novel combination of ingredients, but also from the manner in which they are administered and the apparent inducement of immunological responses that is possible when such materials are provided in the recommended doses and allowed to be absorbed through the epithelial lining of the oral cavity.

The individual components of the composition may be obtained from commercial sources: beta glucan (which is dehydrated by standard spray-drying procedures known in the art) from any processing facility approved by the United States Food and Drug Authority (F.D.A.) such as Biopolymer Engineering, Inc. of St. Paul Minn., U.S.A.; lactoferrin from approved manufacturers such as DMV International Nutritionals of Frazier N.Y., U.S.A.; flavors from approved distributors or manufacturers such as Allen Flavors, Inc. of Edison N.J., U.S.A. Manufacturing of the composition, the dietary supplement, and the oral dosage forms can each be performed using standard techniques appropriate for the food or pharmaceutical industries, as at F.D.A. approved facilities such as Biotics Research, Inc. of Rosenberg, Tex., U.S.A.

### Inventive Compositions

The concentrations of composition ingredients are typically expressed in terms of weight percent. The weight percent of a component is determined based on the weight of the entire composition (i.e. the weight of the component is divided by the total weight of the composition, and multiplied by 100%). For example, 10 mg of beta glucan in a 1000 mg total weight composition would be characterized as 1 weight percent beta glucan.

Generally, the invention is directed towards compositions comprising beta glucan and lactoferrin, and methods for their use. The compositions can be in the form of a liquid, a solid, a capsule, a lozenge, a chewable lozenge, a chewable tablet, a chewable gum, or any other acceptable form. Capsules can be attractive in certain circumstances due to their ease of swallowing. The compositions can be used in baby food preparations. The compositions can be used in enteral feeding preparations where the host is less able to tolerate the fillers and/or flavorings.

Lozenges are presently preferred to be prepared by cold pressing. Lozenges are presently preferred to have a hardness of about 14 Kp to about 35 Kp.

Presently preferred embodiments of the invention include compositions and dietary supplements, as described above, prepared in a “mucosal delivery format”; particularly as an oral dosage form that promotes absorption of the dietary supplement's components through the epithelial lining of the oral cavity. Further preferred embodiments are methods for promoting those beneficial effects in mammals described above, in which such oral dosage forms of these compositions and dietary supplements are administered. Examples of oral dosage forms that promote absorption of the dietary supplement's components within the oral cavity are those that encourage retention of the dose within the oral cavity for an extended period, or discourage swallowing of the dose. Dosage forms that are chewable or that are appropriate for sucking are examples; they can be additionally designed to encourage salivation. Such dosage forms include lozenges, particularly chewable lozenges, chewable tablets and chewable gums. The addition of natural or artificial flavoring also encourages retention of the dosage form within the mouth, particularly with children, so that there is greater transfer of the active components through the lining of the oral cavity and into the bloodstream and/or the lymphatic system. Such active components include the constituents of colostrum and the lactoferrin, as described above. The physical size and consistency of the dosage form can also be adapted to prevent premature swallowing of the delivered dose; 30 seconds to ten minutes is the recommended period for which the dose should remain in the mouth for effective absorption, with better effects being observed at the longer retention times. Larger chewable forms are appropriate for animals that would otherwise be likely to swallow such foodstuff with little mastication.

The compositions can further comprise one or more of the following: nutritionally acceptable carriers, nutritionally acceptable diluents, nutritionally acceptable flavorings, fillers, colorants, binders, and sweeteners. These materials can improve the attractiveness and flavor of the compositions. Examples of these materials include citric acid, sucrose, fruit flavoring, citrus flavoring (such as lemon or orange), silicon dioxide and/or magnesium stearate (as a binder).

The beta glucan can generally be obtained from any source. Presently preferred sources include mushrooms, yeast, and oats. Yeast cell wall beta glucans are a presently more preferred source. The concentration of beta glucan in the compositions can generally be any concentration. The concentration can be about 1 weight percent to about 10 weight percent, about 1 weight percent to about 5 weight percent, or about 1 weight percent to about 2.5 weight percent. A particular composition can have about 1, about 2, about 3, about 4, about 5, about 6, about 7, about 8, about 9, or about 10 weight percent beta glucan.

The lactoferrin can generally be obtained from any source. Presently preferred sources include mammals such as cows (bovine), and more preferably bovine milk. The concentration of lactoferrin in the compositions can generally be any concentration. The concentration can be about 0.25 weight percent to about 2.5 weight percent, about 0.5 weight percent to about 2 weight percent, or about 1 weight percent to about 1.5 weight percent. A particular composition can have about 0.25, about 0.5, about 0.75, about 1, about 1.25, about 1.5, about 1.75, about 2, about 2.25, or about 2.5 weight percent lactoferrin.

A particular composition can be characterized with the concentration of beta glucan in the composition being about 1 weight percent to about 10 weight percent, and the concentration of lactoferrin in the composition being about 0.25 weight percent to about 2.5 weight percent. An additional particular composition can comprise about 1 weight percent beta glucan to about 3 weight percent beta glucan, about 0.5 weight percent lactoferrin to about 1.5 weight percent lactoferrin, and about 5 weight percent nutritionally acceptable flavoring to about 7 weight percent nutritionally acceptable flavoring. A further particular composition can comprise about 2 weight percent beta glucan, about 1 weight percent lactoferrin, and about 5.7 weight percent nutritionally acceptable flavoring.

A further particular composition can consist essentially of, or can consist of the following components: about 2 weight percent beta glucan, about 1 weight percent lactoferrin, about 5.7 weight percent lemon flavoring, about 50 weight percent mannitol, about 40.8 weight percent sorbitol, and about 0.5 weight percent silicon dioxide.

### Inventive Methods

Any of the above described compositions can be used in the following methods.

The above described compositions can be used in methods of treating an individual afflicted with cancer, treating an individual infected with bacteria, treating an individual infected with a fungus, treating an individual infected with a virus, and treating an individual afflicted with septicemia. The compositions also can be used as in a method to prevent or delay the onset of a bacterial infection, fungal infection, viral infection, or septicemia in an individual. The individual is preferably a mammal. The mammal can generally be any mammal such as a human, dog, cat, cow, horse, pig, goat, bear, or moose. The mammal is preferably a human.

Administering the compositions to the individual preferably reduces the amount of cancer, amount of bacteria, amount of fungi, amount of virus, or amount of sepsis in the individual. The reduction is determined by comparing the post-administration amount to the pre-administration amount. The reduction is preferably at least about 10%, at least about 20%, at least about 30%, at least about 40%, at least about 50%, at least about 60%, at least about 70%, at least about 80%, at least about 90%, and ideally about 100%. Prevention of the onset of a bacterial infection, fungal infection, viral infection, or septicemia is relative to the time of onset of a similar individual who was not administered the composition. The delayed onset time is preferably at least about 1 day, 1 week, 1 month, 1 year, and ideally the onset would not happen to the administered individual in a clinically relevant timeframe.

In a presently preferred embodiment of the invention, the composition is taken as a nutritional supplement one to three times per day. The lozenge can be chewed for about 30 seconds to about ten minutes to maximize absorption of the active ingredients through the lining of the oral cavity and their absorption into the blood and lymphatic system.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Preparation of Lozenge Formulation

Preparation of the lozenges was performed with precautions against exposure to the powders and dusts that are formed, and particularly against their inhalation. Each of the following powdered ingredients was placed into a commercial mixer: 20 parts beta glucan, 10 parts bovine lactoferrin, 500 parts mannitol, 408 parts sorbitol, 57 parts natural lemon flavor, and 5 parts silicon dioxide. If necessary, the materials were passed through a #10-12 mesh screen to remove aggregates. After 20 minutes of thorough mixing, cold pressing the composition in a tablet press set at a maximum pressure of 6.4 tons yielded lozenges of weight 1000 mg and hardness 25 to 28 Kp. The lozenge can be characterized as containing 2 weight percent beta glucan, 1 weight percent lactoferrin, 50 weight percent mannitol, 40.8 weight percent sorbitol, 5.7 weight percent lemon flavor, and 0.5 weight percent silicon dioxide.

### Example 2

### Preparation of Capsule Formulation

Each of the following powdered ingredients were placed into a commercial mixer following the same procedure as described in Example 1, except they were encapsulated in a #2 gelatin capsule: 20 parts beta glucan, 10 parts bovine lactoferrin, and 270 parts filler. After mixing and encapsulating, capsules of weight 300 mg were formed. The lozenge can be characterized as containing about 6.7 weight percent beta glucan, about 3.3 weight percent lactoferrin, and about 90 weight percent filler.

### Example 3

### Effects of Lozenges on Sinus Infections

A human female with acute sinusitis and sinus infection sought medical care. Consultation with her physician resulted in the first of a series of prescriptions for antibiotics. After taking the first treatment, a return visit to the physician showed no improvement. A second type of antibiotics was prescribed, again with no improvement. After taking the second treatment without beneficial effect, a third antibiotic was prescribed. After eight weeks without noticeable improvement, the patient started a regimen of taking mucosally delivered lozenges containing 20 mg beta glucan and 10 mg lactoferrin three times per day. After three days of the regimen, the patient experienced considerable relief from inflammation. By the end of the fifth day of the regimen, the infection had been eliminated and the patient had no discomfort or distress.

### Example 4

### Effects of Lozenges on Terminal Cancer

A human male patient with terminal pancreatic and liver cancers was discharged from further hospital treatment and was allowed to go home to die. Hospice was called to provide the final in home health treatment, which consisted of monitoring and overseeing the administration of a morphine pump. The patient's wife started him on a regimen of taking mucosally delivered lozenges containing 20 mg beta glucan and 10 mg lactoferrin three times per day. The wife would place a lozenge in his mouth during his brief periods of consciousness. The formulation of the lozenge dissolved easily to provide mucosal delivery. After two weeks of the regimen, the patient was feeling well enough to be taken off of the morphine pump and had resumed some household functions such as driving a car and shopping at the grocery store. Twenty-six days after being taken off of the pump, the cancerous organs finally shut down and the patient died.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

What is claimed is:

1. A composition comprising beta glucan and lactoferrin.

2. The composition of claim 1, wherein the composition is a solid.

3. The composition of claim 1, wherein the composition is in the form of a capsule, lozenge, chewable lozenge, tablet, chewable tablet, or chewable gum.

4. The composition of claim 1, wherein the composition is a liquid.

5. The composition of claim 1, wherein the beta glucan is mushroom beta glucan, yeast beta glucan, or oat beta glucan.

6. The composition of claim 1, wherein the beta glucan is yeast cell wall beta glucan.

7. The composition of claim 1, wherein the concentration of beta glucan is about 1 weight percent to about 10 weight percent.

8. The composition of claim 1, wherein the lactoferrin is mammalian lactoferrin.

9. The composition of claim 1, wherein the lactoferrin is bovine milk lactoferrin.

10. The composition of claim 1, wherein the concentration of lactoferrin is about 0.25 weight percent to about 2.5 weight percent.

11. The composition of claim 1, wherein:
the concentration of beta glucan is about 1 weight percent to about 10 weight percent; and
the concentration of lactoferrin is about 0.25 weight percent to about 2.5 weight percent.

12. The composition of claim 1, further comprising at least one nutritionally acceptable carrier.

13. The composition of claim 1, further comprising at least one nutritionally acceptable diluent.

14. The composition of claim 1, further comprising at least one nutritionally acceptable flavoring.

15. The composition of claim 1, wherein the composition is prepared in an oral dose specific format that promotes absorption of the composition's components within the mucosal layer in the oral cavity.

16. A composition comprising:
about 1 weight percent beta glucan to about 3 weight percent beta glucan;
about 0.5 weight percent lactoferrin to about 1.5 weight percent lactoferrin; and
about 5 weight percent nutritionally acceptable flavoring to about 7 weight percent nutritionally acceptable flavoring.

17. A composition comprising:
about 2 weight percent beta glucan;
about 1 weight percent lactoferrin; and
about 5.7 weight percent nutritionally acceptable flavoring.

18. A composition consisting essentially of:
about 2 weight percent beta glucan;
about 1 weight percent lactoferrin;
about 5.7 weight percent lemon flavoring;
about 50 weight percent mannitol;
about 40.8 weight percent sorbitol; and
about 0.5 weight percent silicon dioxide.

19. A method of treating an individual afflicted with cancer, the method comprising administering to the individual a composition comprising beta glucan and lactoferrin.

20. A method of treating an individual infected with bacteria, the method comprising administering to the individual a composition comprising beta glucan and lactoferrin.

21. A method of treating an individual infected with a fungus, the method comprising administering to the individual a composition comprising beta glucan and lactoferrin.

22. A method of treating an individual infected with a virus, the method comprising administering to the individual a composition comprising beta glucan and lactoferrin.

23. A method of treating an individual afflicted with septicemia, the method comprising administering to the individual a composition comprising beta glucan and lactoferrin.

24. A method of preventing or delaying the onset of septicemia in an individual, the method comprising administering to the individual a composition comprising beta glucan and lactoferrin, whereby the onset time of septicemia is longer than the onset time of a similar individual to whom the composition is not administered.
